(19) United States
(12) Patent Application Publication
(10) Pub. No.: US 2009/0099553 A1
(43) Pub. Date: Apr. 16, 2009

(54) DEVICE FOR THE CONTROLLED RELEASE OF A SUBSTANCE AND METHOD OF RELEASING A SUBSTANCE

(75) Inventors: Geert Langereis, Eindhoven (NL); Ventzeslav Petrov Iordanov, Valkenswaard (NL); Michel Paul Barbara Van Bruggen, Helmond (NL); Hendrika Cecilia Krijnsen, Boxtel (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJDE PHILIPS ELECTRONICS N.V., EINDHOVEN (NL)

(21) Appl. No.: 12/298,069

(22) PCT Filed: Apr. 17, 2007
(86) PCT No.: PCT/IB2007/051375

§ 371 Date: Oct. 22, 2008

(30) Foreign Application Priority Data
Apr. 26, 2006 (EP) 06113138.9

(51) Int. Cl. A61K 9/22; B05D 1/36; A61F 7/00; A61M 31/00

(52) U.S. Cl. 604/891.1; 427/2.14; 604/114; 604/500

(57) ABSTRACT

This invention provides a device for the controlled release of a substance and a method of controllably releasing a substance from a compartment. The device comprises a plurality of compartments in a substrate, each compartment being closed by at least one release mechanism, wherein the release

The present invention relates a device for the controlled release of a substance. The present invention further relates to a method for controllably releasing a substance from a compartment.

Accurate delivery of small, precise quantities of one or more chemicals into a carrier fluid are of great importance in many different fields of science and industry. Examples in medicine include the administering drugs to patients using intravenous methods, by pulmonary or inhalation methods or by the release of drugs from vascular stent devices. Examples in diagnostics include releasing reactions into fluids to conduct DNA or genetic analysis, combinatorial chemistry, or the detection of a specific molecule in an environmental sample. Other applications involving the delivery of chemicals into a carrier fluid include the release of fragrances and therapeutic aromas from devices into air and the release of flavoring agents into a liquid to produce beverage products.

Devices for the controlled release of a predefined quantity of a substance are generally known. For example, US patent application US 2004/0034332 A1 discloses an implantable device for controlled delivery of a drug, the device including a microchip which has reservoirs containing the molecules for release. The microchip device includes a substrate, at least two reservoirs in the substrate containing the molecules for release and a reservoir cap positioned on or within a portion of the reservoir and over the molecules, so that the molecules are controllably released from the device by diffusion through or upon disintegration or rupture of the reservoir caps. Each of the reservoirs of a single microchip can contain different molecules which can be released independently. One drawback of the known device is that the reservoir cap is burnt away, and, consequently, the removed material ending up in the human body. Furthermore, close to the reservoir cap, which is usually a membrane, the temperature will be comparably high while the reservoir cap is burnt away. This might deteriorate the substance to be released, especially in the case of drugs to be administered for medical reasons. A further drawback is that the opening of the reservoir is irreversible, i.e. once it is opened, it cannot be closed again.

It is therefore an object of the present invention to provide a device for the controlled release of a substance that can be more flexibly used.

The above objective is accomplished by a device and a method for the controlled release of a substance according to the present invention, wherein the device comprises a plurality of compartments in a substrate, each compartment being closed by at least one release mechanism, wherein the release mechanism is provided openable and reclosable.

An advantage of the device according to the invention is that it is possible to implement a controlled substance or drug delivery system based upon a multiplicity of individual drug release compartments, where such a device has a wide range of applications and wherein the control requirements as well as the manufacturing requirements and, consequently, the costs of the device are reduced. According to the prior art, a compartment is either closed or it is completely opened. Consequently, there is neither an intermediate state in which the compartment is only partly opened noris there the possibility of reclosing the compartment.

A further advantage of the present invention is that thereby, applications as for example external drug delivery systems (patches), implantable drug delivery systems or oral drug delivery systems (e-pill) are possible. A drug delivery system according to the present invention may be applied for delivery of a single drug but can be advantageously applied to a system in which several different drugs are applied from the same arrangement of compartments or the same device.

According to the present invention, it is very much preferred that the release mechanism is provided partly openable. Thereby it is possible to achieve a so called basal release rate of the substance in the compartment where such a basal release rate is lower than a regular (or bolus) release rate with the compartment completely opened. This may be extremely beneficial to special treatments, e.g. of diabetes, where the use of a basal rate and a bolus rate is very useful. Another example is pain relief, having a constant low amount of pain relieving medication at all times of day (basal rate) and activate the valve (bolus) when pain perception increases.

It is furthermore preferred that the release mechanism comprises at least a first material and a second material, the first material having a first thermal expansion coefficient, the second material having a second thermal expansion coefficient, wherein the opening and/or the closing of the compartment is provided upon heating and/or cooling of the first and second material. Thereby, it is possible to realise a very stable and robust device able to being repeatedly actuated by means of a change in temperature.

Furthermore, it is preferred that the device comprises a means for heating and/or cooling the release mechanism. Thereby, a simple way of actuation of the device by means, for example, of an electrical resistor or a thermoelement is possible.

According to the present invention, it is very much preferred that the release mechanism is provided as a lid covering at least one of the plurality of compartments or an opening of at least one of the plurality of compartments. Thereby, it is possible to realize the advantages of the present invention by means of an arrangement, which is very easy to manufacture. Especially, it is very easy to provide a lid as a release mechanism and to provide the first and second material as a part of the lid.

It is furthermore preferred that the lid comprises a first portion fixed to the substrate and a second portion movable relative to the substrate. Thereby, a valve-like movement of the lid or at least of a part of the lid can be provided.

Furthermore, it is preferred that the lid or the release mechanism comprises a sealing means such that the sealing means is broken upon a movement of the second portion of the lid relative to the substrate. The sealing means is very advantageously provided in order to ensure a tight closure of the compartment before use. Consequently, between the manufacturing of the compartments and the use of the substance to be released, no substance can be lost.

According to the present invention, it is furthermore very much preferred that the sealing means is a seal layer or at least a part of a seal layer between the substrate and the first and/or the second material. Thereby, an inert or insulating material can be chosen for containing the substance, especially a drug, so that there are no risks of contamination of the substance.

It is furthermore preferred that the sealing means is part of the first and/or the second material. Thereby, a very cost-effective device can be realised which can moreover be manufactured very easily and rapidly.

Furthermore, it is preferred that the lid comprises a means for heating and/or cooling the lid. Thereby, it is possible to actuate the lid very easily and very rapidly.

In another embodiment of the present invention, it is preferred that the means for heating and/or cooling the lid is provided exclusively on the first portion of the lid. This has the advantage that the means for heating and/or cooling is not bent during the movement of the second portion of the lid.

According to the present invention, it is also preferred that the means for heating and/or cooling the lid is provided on the second portion of the lid or on the first portion and on the second portion of the lid. Thereby, the heat or the decrease in temperature can be applied very effectively to the interface between the first material and the second material.

It is furthermore preferred that the difference in temperature to be applied by the means for heating and/or cooling the lid is at least 10 degrees centigrade, preferably at least 20 degrees centigrade. This enables the lid to stay closed even in the event of local and minor temperature peaks, thereby enabling the compartments to stay closed even when transported or otherwise treated.

In a still further preferred embodiment of the present invention, a first group of compartments is provided to contain a first quantity of a first substance and a second group of compartments is provided to contain a second quantity of a second substance. An advantage of the device according to the present invention is that a very flexible substance release mechanism can be implemented in the structure of the inventive device. For example, it is possible to provide compartments of different size, thereby being able to contain different volumes of the substance or substances to be released. For example, if at a given moment a greater quantity of a substance is to be released, the device can be controlled accordingly and open a compartment having an appropriate size and hence an appropriate volume of the substance to be released. This is instead of releasing the same quantity of substance from a certain number of smaller compartments which would have the same effect. Of course, the release of an appropriate quantity of a substance from a single compartment is easier to control and therefore makes the device according to the present invention smaller, more lightweight and more cost-effective. Accordingly, the first and the second substance can be different or identical. Another way of improving the flexibility of releasing substances like drugs or the like is providing several different substances or different mixtures of substances in different compartments on the device, the different compartments being of the same or a different size. It is thereby possible to controllably release to the patient for example two different drugs alternately during the day or during another time interval. Alternatively, it is also possible to further enhance the flexibility of use of the inventive device for example by providing differently sized compartments as well as different substances in the differently sized compartments. It is preferred according the present invention, that the first quantity is approximately half the second quantity. It is thereby also possible to have a first group of compartments having a first volume or containing a first quantity of a substance, a second group of compartments containing each twice the first quantity, a third group containing four times the first quantity and a fourth group of compartments containing eight times the first quantity. Thereby flexibility of releasing one or more substances is even further enhanced.

Further embodiments of the present invention are provided with a control unit for controlling the release of the substance. It is further preferred, that the first number is at least 10, preferably at least 100, more preferably at least 1,000, still preferably at least 10,000 compartments. The compartments can be filled by micropipette or ink jet printing techniques or as a whole from the back before the substrate is sealed on the backside (only for the case of all identical molecules in the compartments).

The present invention also includes a method for controllably releasing a substance from a compartment using a device that comprises a plurality of compartments in a substrate, each compartment being closed by at least one release mechanism, wherein the release mechanism is provided openable and reclosable, the method comprising the step of:
applying an actuation signal to the release mechanism, thereby opening the release mechanism.

It is thereby possible to controllably release a specific quantity of a substance in a very rapid and easily controlled manner. Furthermore, energy has only to be applied for the opening of the release mechanism.

In a preferred embodiment of the method according to the present invention, the actuation signal provides for a heating and/or cooling of the release mechanism, preferably a difference in temperature applied to the release mechanism of at least 10 degrees centigrade, preferably of at least 20 degrees centigrade. Thereby, the control of the inventive device can be performed very easily and by means of only a small amount of energy.

In a preferred embodiment of the method according to the present invention, more than one compartment release the substance at the same time. This may mean that more than one compartment are opened simultaneously and that the period of releasing the substance or the drug is then collective for each of these compartments. Alternatively, it is also possible that a plurality of compartments are opened sequentially so that their periods of release (usually much longer than the time required for opening a specific compartment) overlap and a release of the substance by more than one compartments is possible. It is thereby possible to very flexibly control the release of a substance.

The present invention also includes a method of manufacturing an inventive device for controllably releasing a substance from a compartment using a device that comprises a plurality of compartments in a substrate, each compartment being closed by at least one release mechanism, wherein the release mechanism is provided openable and reclosable, the method comprising the step of depositing or creating a first material and a second material on a substrate for forming the release mechanism.

It is thereby possible to produce the inventive device in a very rapid and cost effective manner.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

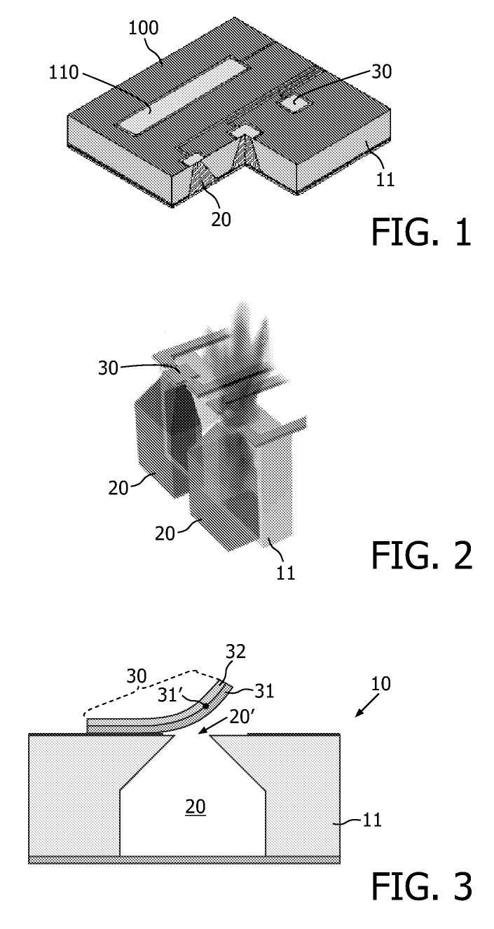

FIGS. 1 and 2 illustrate schematically a device 100 according to the prior art showing in principle a structure of a device of such a type.

FIGS. 3 to 5 illustrate schematically two embodiments of a part of a device 10 of the present invention.

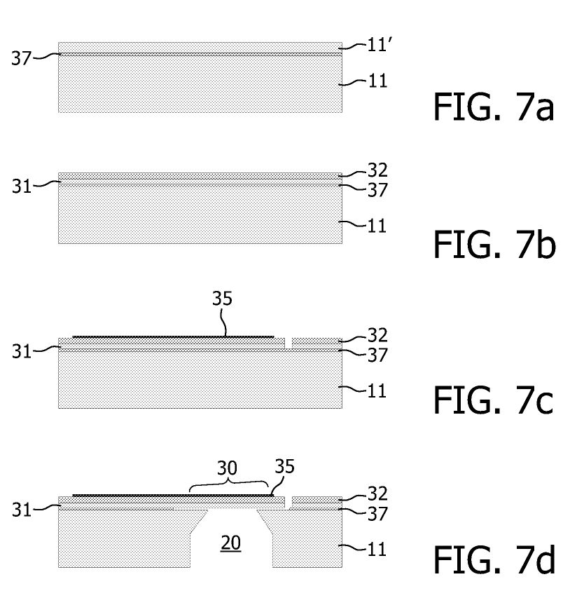

FIGS. 6 and 7 illustrate schematically different methods for manufacturing a device according to the present invention.

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn to scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operating in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operating in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIGS. 1 and 2, a known device 100 according to the prior art is schematically shown. The known device 100 comprises a substrate 11 where a plurality of compartments 20 are located. The compartments 20 are closed by a release mechanism 30, especially a closure cap 30. It can further be seen from FIG. 1 that there are electrode lines running to each of the compartments 20 or at least to or near to each of the release mechanisms 30. The connecting lines are not described with a reference sign in FIG. 1. The known device 100 further comprises an electrode area 110. FIG. 2 shows the case of an intact release mechanism 30 and also the case of an actuated (or “blown”) release mechanism 30 or closure cap 30 in order to disperse or release a substance, especially a drug.

In FIGS. 3 to 5 schematical representations of a part of an inventive device 10 are shown according to two embodiments of the present invention. In FIGS. 3 to 5, for reasons of simplicity, only one compartment 20 is shown, but it is evident, that the inventive device 10 comprises a plurality of compartments 20, e.g. at least 100 compartments, preferably at least 1,000 compartments and most preferably at least 10,000 compartments. The device 10 comprises the compartments 20 in a substrate 11 comparable to the prior art devices. The substrate 11 is the structural body in which the compartments 20 are formed, e.g. it contains the etched, machined or molded compartments 20. A compartment 20 (which is also called a reservoir in the following) is a container for a substance. Micro-electromechanical system methods, micro-molding and micro-machining techniques known in the art can be used to fabricate the substrate 11 together with the compartments 20 from a variety of materials. Examples of suitable substrate materials include metals, ceramics, semiconductors, degradable and non-degradable polymers. Bio-compatibility of the substrate material is typically preferred for in-vitro device applications. The substrate, or portions thereof, may be coated, capsulated, or otherwise contained in a bio-compatible material before use. The substrate 11 can be flexible or rigid. In one example, the substrate 11 is formed of silicon or another semiconductor material. The substrate 11 can have a variety of shapes for shaped surfaces. It can, for example, have a release side, i. e. an area having release mechanisms, that is planar or curved. The substrate may for example be in a shape selected from discs, cylinders, or spheres. In one embodiment, the release side can be shaped to conform to a curved tissue surface. This would be particularly advantageous for local delivery of a therapeutic agent to that tissue surface. In another embodiment the backside (distal to the release side) is shaped to conform to an attachment surface. The substrate 11 may consist of only one material or may be a composite or multi-laminate material, that is, composed of several layers of the same or different substrate materials that are bonded together.

In FIG. 3 a single compartment 20 is shown as part of the device 10 according to the present invention. The compartment 20 is located in the substrate 11 with a release mechanism 30. The release mechanism 30 is formed as a lid 30 covering one opening 20′ of the compartment 20 (on the release side of the device 10). The compartment 20 can be provided with a second opening on the other side (distal to the release side) of the substrate 11. This second opening is normally closed and only opened in order to fill the compartment 20 with the substance to be released by the inventive device 10. The lid 30 comprises a first material 31 and a second material 32. The two materials 31, 32 are facing each other along an interface 31′ or contact surface 31′. According to the present invention, the first material 31 has a different thermal expansion coefficient from the second material 32. A heating of the stack of the first and second material 31, 32 will result in a bending of the stack of the first and the second material 31, 32. This allows for the realization of a valve mechanism or release mechanism 30 at the opening 20′ of the compartment 20. When a difference of temperature is applied to the closure lid 30, the bending of the first and the second material 31, 32 can be controlled and therefore the opening or the closing of the opening 20′ of the compartment 20 can be performed. Inside the compartment 20 is located a substance, preferably a drug or another substance to be released in a very controlled manner.

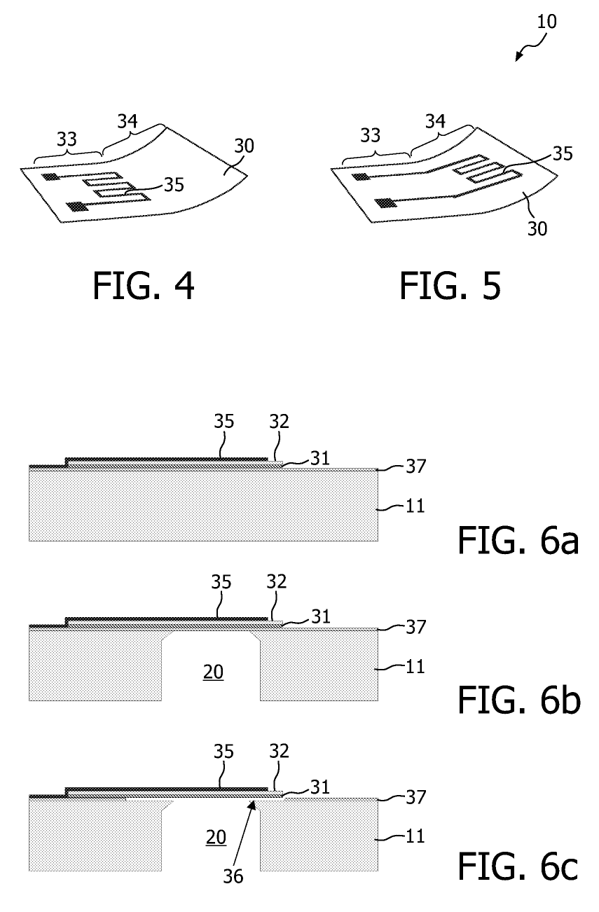

In FIGS. 4 and 5 there are two different embodiments of a closure lid 30 or id according to a device 10 of the present invention. The lid 30 comprises a first portion 33 which is fixed relative to the substrate 11 (not shown in FIGS. 4 and 5) and a second portion 34 which is movable relative to the substrate 11. In order to apply a difference in temperature to the lid 30, a means for heating and/or cooling the release mechanism 30 is preferably provided. Such a means for heating and/or cooling is designated by reference sign 35. In the first embodiment (represented in FIG. 4), the means for heating and/or cooling 35 is located only in the first portion 33 of the release mechanism 30. In the second embodiment of the present invention (represented in FIG. 5), the means for the heating and/or cooling 35 is located also in the second portion 34 of the release mechanism 30. Furthermore, the means for heating and/or cooling 35 has contact paths which are also located in the first portion 31 of the means for heating and/or cooling 35.

In FIGS. 6 and 7, schematical illustrations of different methods of manufacturing a device according to the present invention are shown.

In FIG. 6, a first example of a manufacturing method of the device 10 is shown. In a first step (cf. FIG. 6a), on a material for forming the substrate 11, preferably a silicon substrate or more generally a semiconductor substrate, a Silicon Nitride layer (Si₃N₄) is deposited especially by an LPCVD method. This layer creates the DC insulation for the electrodes of the means for heating and/or cooling 35 of the lid 30 as well as for the first and second material 31, 32. This insulation layer is represented by reference sign 37. On the insulation layer 37, the first material 31 and the second material 32 are deposited. For example, the first material can be an aluminum layer and the second material can be a gold layer. On top of the second material 32, the means for heating and/or cooling can be located, especially by means of a tungsten resistance or a platinum resistance. The thermal expansion coefficient of aluminum is 22·10⁻⁶ K⁻¹. The thermal expansion coefficient of gold is 14·10⁻⁶ K⁻¹. The thermal expansion coefficient of tungsten is 4.5·10⁻⁶ K⁻¹. The thermal expansion coefficient of platinum is 8.9·10⁻⁶ K⁻¹. In a second step, represented in FIG. 6b, the capacity of the compartment 20 is etched from the backside (distal to the release side of the opening 20′) of the substrate 11 using an isotropic and/or an anisotropic etching step or an isotropic and, subsequently, an anisotropic etching step. This etching step or these etching steps can be performed, e.g., through a Silicon Nitride mask (not shown) on the backside of the substrate 11. Finally, the valve or the lid 30 is released by a Nitride etching step from the backside in step three (cf. FIG. 6c).

Thereby a small gap between the valve or the lid 30 and the substrate 11 is realized, which might lead to a leaking out of the substance. To avoid this, the stack of materials should be chosen such that due to a stress difference in the different material layers, the lid 30 pushes downwards towards the substrate 11. Alternatively, a weak residual link between the lid 30 and the substrate 11 can be provided (e.g. in the insulation layer 37 then serving as a sealing means 36) which is broken when the lid is actuated. In this case, the system is irreversible (with respect to the sealing means 36). The residual link is also called the sealing means 36 and is located near the movable end (second portion 34) of the lid 30. In FIG. 6c only an arrow points to the location of such a sealing means 36.

In FIG. 7, a second example of a manufacturing method of the device 10 is shown. In this process, a SOI-wafer (Silicon on Insulator) is used as the substrate 11. The top polysilicon layer 11′ is used therewith, at least partly, as the first material 31, whereas the intermediate silicon oxide material is used as the insulation layer 37. Both are located on one side of a SOI substrate, which is represented in FIG. 7a.

In FIG. 7b a silicon dioxide layer is applied to the SOI wafer. The silicon dioxide is used as the second material 32. The silicon dioxide layer on the SOI wafer can be realized either by depositing or by means of thermally oxidizing a part of the polysilicon layer 11′ of the SOI wafer. The latter alternative is shown in FIG. 7b where the SiO₂ layer forms the second material 32 and where the rest of the polysilicon layer 11′ forms the first material 31. On top of the silicon dioxide layer 32 is deposited a heating element 35 to realize the means for heating and/or cooling the lid 30, as shown in FIG. 7c. In FIG. 7d the creation of the compartment 20 from the backside of the substrate 11 is shown. There the silicon oxide intermediate layer (insulating layer 37) serves as an etch stop layer.

According to the present invention it is also possible to provide the second material 32 to be electrically insulated with respect to the first material 31 and thereby serving both as the second material 32 and as a heating element 35. It is also possible according to the present invention, to provide both the first and the second material 31, 32 to be heating elements. Furthermore, if the first material 31 is provided to be insulating if the substrate 11 material is provided to be insulating, it is also possible that the first material also serves as insulating material.

1. Device (10) for the controlled release of a substance, the device (10) comprising a plurality of compartments (20) in a substrate (11), each compartment (20) being closed by at least one release mechanism (30), wherein the release mechanism (30) is provided openable and reclosable.

2. Device (10) according to claim 1, wherein the release mechanism (30) is provided partly openable.

3. Device (10) according to claim 1, wherein the release mechanism (30) comprises at least a first material (31) and a second material (32), the first material (31) having a first thermal expansion coefficient (41), the second material (32) having a second thermal expansion coefficient (42), wherein the opening and/or the closing of the compartment (20) is provided upon heating and/or cooling of the first and second material (31, 32).

4. Device (10) according to claim 3, wherein the device (10) comprises a means (35) for heating and/or cooling the release mechanism (30).

5. Device (10) according to claim 3, wherein the release mechanism (30) is provided as a lid (30) covering at least one of the plurality of compartments (20).

6. Device (10) according to claim 5, wherein the lid (30) comprises a first portion (33) fixed to the substrate (11) and a second portion (34) movable relative to the substrate (11).

7. Device (10) according to claim 6, wherein the lid (30) comprises a sealing means (36) such that the sealing means (36) is broken upon a movement of the second portion (34) of the lid (30) relative to the substrate (11).

8. Device (10) according to claim 7, wherein the sealing means (36) is a seal layer (36) between the substrate (11) and the first and/or the second material (31, 32).

9. Device (10) according to claim 7, wherein the sealing means (36) is part of the first and/or the second material (31, 32).

10. Device (10) according to claim 5, wherein the lid (30) comprises a means (35) for heating and/or cooling the lid (30).

11. Device (10) according to claim 10, wherein the means (35) for heating and/or cooling the lid (30) is provided exclusively on the first portion (33) of the lid (30).

12. Device (10) according to claim 10, wherein the means (35) for heating and/or cooling the lid (30) is provided on the second portion (34) of the lid (30) or on the first portion (33) and on the second portion (34) of the lid (30).

13. Device (10) according to claim 10, wherein the difference in temperature to be applied by the means (35) for heating and/or cooling the lid (30) is at least 10 degrees centigrade.

14. Device (10) according to claim 10, wherein the difference in temperature to be applied by the means (35) for heating and/or cooling the lid (30) is at least 20 degrees centigrade.

15. Method of controllably releasing a substance from at least a compartment (20) using a device (10) comprising a plurality of compartments (20) in a substrate (11), each compartment (20) being closed by at least one release mechanism (30), wherein the release mechanism (30) is provided openable and reclosable, the method comprising the step of:
applying an actuation signal (45) to the release mechanism (30), thereby opening the release mechanism (30).

16. Method according to claim 15, wherein the actuation signal (45) provides a heating and/or a cooling of the release mechanism (30).

17. Method according to claim 16, wherein the difference in temperature applied to the release mechanism (30) is at least 10 degrees centigrade.

18. Method according to claim 16, wherein the difference in temperature applied to the release mechanism (30) is at least 20 degrees centigrade.

19. Method of manufacturing a device (10) for the controlled release of a substance, the device (10) comprising a plurality of compartments (20) in a substrate (11), each compartment (20) being closed by at least one release mechanism (30), wherein the release mechanism (30) is provided openable and reclosable, wherein the method comprises the step of:
depositing or creating a first material (31) and a second material (32) on a substrate (11) for forming the release mechanism (30).

20. Method according to claim 19, wherein an insulating layer (37) and/or a means (35) for heating and/or cooling is deposited or created under and/or on top of the first and the second material (31, 32).
